(19) United States
(12) Patent Application Publication
(10) Pub. No.: US 2017/0224962 A1
(43) Pub. Date: Aug. 10, 2017

(54) Safety Guidewire Introducer Assembly

(71) Applicant: M. Samy Ahmed Hamboly, 10th of Ramadan City (EG)

(72) Inventor: M. Samy Ahmed Hamboly, 10th of Ramadan City (EG)

(21) Appl. No.: 15/016,538

(22) Filed: Feb. 5, 2016

(51) Int. Cl. A61M 25/06

(52) U.S. Cl. CPC A61M 25/0618; A61M 25/0631; A61M 25/09041

(57) ABSTRACT

The catheter introducer assembly comprises an outer tube, an outer tube housing, a needle, a needle extension member, a spring, and a retainer portion. The outer tube nests within the outer tube housing. The needle, needle extension member, and the, spring nest within the outer tube and outer tube housing. The needle tip is moved from the first position to a second position upon connection of a male luer slip to the retainer portion and the needle tip is moved from the second position to the first upon decoupling.

### FIELD OF THE INVENTION

The present invention relates generally to catheter introducer needles and specifically to a safety guidewire introducer assembly in which a needle tip is protected by a protective shield that reduces the risk of accidental needle stick injuries. The assembly comprises a protective tube that retains the needle tip until connected to a syringe male luer slip. The needle tip is automatically retracted back into the protector shield once the syringe male luer is disconnected.

### BACKGROUND OF THE INVENTION

Medical care of individuals requires the widespread use of catheter devices for taking blood samples, for intravenous (IV) drug delivery, and for the introduction or removal of other fluids. The use of a catheter device typically involves first inserting a needle into the vein of a patient and then advancing a guidewire through it then removing, the needle to leave only the guidewire in the vein where a catheter can be advanced into the vein over such wire. The withdrawn needle is then typically disposed of. The disposed needle presents a risk of a needle stick that might injure health care workers. A needle stick can result in a transfer of a potential of infection such as Acquired Immune Deficiency Syndrome (AIDS), hepatitis, or other conditions which can be transmitted through the exchange of body fluids by accidental contact with the needle tip of a used needle. Therefore, needle stick protection has never been more important.

### DESCRIPTION OF THE PRIOR ART

Commercially available safety catheter devices generally may be grouped into three basic categories: (1) those which include an activation mechanism to hide the withdrawn needle within a needle shield; (2) those which require placement of a separate needle guard; and (3) those which include a sliding shield which must be manually positioned distally over the used needle. Despite laudable intentions, most of these safety catheter devices suffer from shortcomings. Among other things, some safety catheter devices require an extra step beyond the normal insertion and withdrawal steps and/or require additional complicated structural elements to ensure that the needle guard arid the catheter remain attached during the withdrawal procedure.

Of the first category, there are several different conventional systems available with a common design which utilizes a frictionally locked spring biased needle shield to automatically engage the needle tip when the needle is withdrawn from the catheter. While these devices may be considered adequate, a user can unknowingly apply sufficient force during the withdrawal process to overcome the frictional engagement in the device so that the spring biased shield can slip off of the distal end of the needle and leave the needle tip exposed. Extending shields have the burden of additional length, increasing the need for additional space when using the device. Other designs provide retractable devices that may require considerable additional length, as compared to a traditional non-safety device, to provide a concealment chamber for the contaminated piercing member. Another disadvantage of the retractable device is the necessity to manually activate the safety feature. The imposing length and additional manipulation of this system make retractable systems unsuitable for directly replacing, non-safety medical devices.

In conventional prior art devices of the second and third categories, there are, a number of different designs which include needle shields with either a spring-clip fitting or a frictional fitting. A needle shield of this type is either placed directly on the needle tip or is movable from the base of the needle to the tip of the needle along the longitudinal direction of the needle. In the latter type, a user can manually slide the needle shield towards the needle tip to engage the needle shield thereto. However, these manually activated designs require that the user either slide or apply the needle shield to the tip of the needle by hand, thus significantly raising the risk of unintentional contact with the needle tip.

In another prior art catheter assembly, a safety guard is configured to sit in the catheter hub until the needle is withdrawn. Once the needle is withdrawn, the safety guard automatically unlocks from the catheter hub to block the needle tip. This design, however, adds an inherent adverse drag between the needle and the safety guard as the needle is withdrawn. This drag is undesirable because it can create an uneven withdrawal movement

More recently, a catheter introducer assembly was designed that automatically moves the needle spring clip into position to block the needle tip upon detachment of the needle from the catheter body. This action occurs as a direct consequence of the withdrawal of the needle from the catheter bore, without the need for the activation of any additional mechanism to complete the needle withdrawal process. Although it is an automatic (passive) mechanism it still does not reduce the risk of early disengagement or miss-engagement.

Accordingly, there remains a need for a catheter assembly which reduces the risk of early disengagement by both holding the needle shield and the catheter body together during the withdrawal process and simultaneously and automatically disengaging from the catheter body with substantially zero adverse drag and blocking of the needle tip once the withdrawal process is complete.

### SUMMARY OF THE INVENTION

The catheter introducer assembly according to the present invention offers an automatic protrusion of a needle tip out of its shield once a syringe is connected to the assembly. The needle tip is automatically retracted back into the shield once the syringe is disconnected.

The catheter introducer assembly comprises an outer tube, an outer tube housing, a needle, a needle extension member, a spring, and a retainer portion. The outer tube comprises a blunt distal end. At a proximal end, the outer tube is coupled to the outer tube housing. The outer tube housing comprises a tubular funnel-shaped configuration such that the outer tube housing comprises a narrow distal portion, a tapering mid-portion and a wider proximal portion. The narrow distal portion comprises an inside diameter slightly larger than an outside diameter of the outer tube such that the proximal end of the outer tube nests within the narrow distal portion of the outer tube housing.

Nesting within the outer tube and outer tube housing are the needle, needle extension member, and the spring. The needle, is generally tubular, and comprises a sharp distal end (needle tip) and a blunt proximal end. A portion of the needle proximate to the blunt proximal end of the needle is coupled to the needle extension member. The needle extension member is tubular and comprises a needle extension member tapering portion and needle extension member retaining portion. The needle extension member tapering portion comprises a widened proximal end and a narrow distal end such that the needle extension member tapering portion narrows in diameter between the proximal end of the widened needle extension member tapering portion and the needle extension member retaining portion. The needle extension member retaining portion comprises an inside diameter slightly larger than an outside diameter of the needle such that the proximal end of the needle nests within the needle extension member retaining portion.

The proximal end of the needle extension member comprises a push plate. Push plate spans between perimeter walls of the needle extension member and serves as a surface against which a male leer can push so as to cause the needle tip to extend beyond the distal end of outer tube. The proximal end of the needle extension member comprises an outside diameter that is slightly smaller than an inside diameter of the outer tube housing wider proximal portion. Thus, the needle, together with the attached needle extension member can freely move, coaxially, within the outer tube housing wider proximal portion. The spring of the preferred embodiment is a coil spring. The spring is positioned around the perimeter of the needle extension member and a portion of the proximal end of the needle and extends between the outer tube housing wider proximal portion and the tapering mid-portion of the outer tube housing. When in position, the spring, needle, needle extension member, outer tube, and outer tube housing are coaxial.

The retainer portion is coupled to the outer tube housing. The retainer portion is generally tubular in configuration and comprises retainer portion wall and retainer rim. Retainer wall is cylindrical and forms a retainer portion distal inside diameter that is slightly larger than the outside diameter of the wider proximal portion of outer tube housing such that the outer tube housing nests within a distal section of the retainer portion.

The retainer portion wall comprises a retainer portion proximal inside diameter that is smaller than the inside diameter of the outer tube housing. This retainer portion proximal inside diameter is created by thickened walls in an area distal to the outer tube housing. The thickened portions of walls form a ridge against which the push plate rests when in the retracted rest position.

The retainer portion forms a female luer hub adapted to receive a male luer of a syringe. In use, the male luer pressed towards the push plate between the walls of the retainer portion. This action causes the needle tip to extend beyond the distal end of outer tube. Thus, the catheter introducer assembly of the present invention offers automatic protrusion of the needle tip out of the outer tube once a syringe is connected to the assembly. The needle tip automatically retracts back into the outer tube once the syringe is disconnected.

### BRIEF DESCRIPTION OF THE DRAWINGS

FIG. 1 is a cut-away elevation view of a partially assembled safety guidewire introducer assembly, the needle being in a retracted “rest” position, in accordance with a preferred embodiment.

FIG. 1A is an enlarged end view of the safety guidewire introducer assembly of FIG. 1.

FIG. 2 is a cut-away elevation view of an assembled safety guidewire introducer assembly, the needle in a retracted position, in accordance with a preferred embodiment.

FIG. 2A is a close-up of the dashed circled area of FIG. 2.

FIG. 3 is a cut-away elevation view of an assembled safety guidewire introducer assembly together with a syringe comprising a male luer, the needle in an extended position, in accordance with a preferred embodiment.

### DETAILED DESCRIPTION OF THE INVENTION

The present invention in its various embodiments and aspects of such embodiments provides a catheter introducer assembly which reduces the risk of early disengagement by both holding the needle shield and the catheter body together during the withdrawal process and simultaneously and automatically disengaging from the catheter body with substantially zero adverse drag and blocking the needle tip once the withdrawal process is complete.

As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

Reference throughout this document to “one embodiment,” “certain embodiments,” “an embodiment,” or similar term means that a particular feature, structure, or characteristic described in connection with the embodiment is included in at least one embodiment of the present disclosure. Thus, the appearances of such phrases in various places throughout this specification are not necessarily all referring to the same embodiment. Furthermore, the particular features, structures, or characteristics may be combined in any suitable manner on one or more embodiments without limitation.

### Description of the Preferred Embodiment

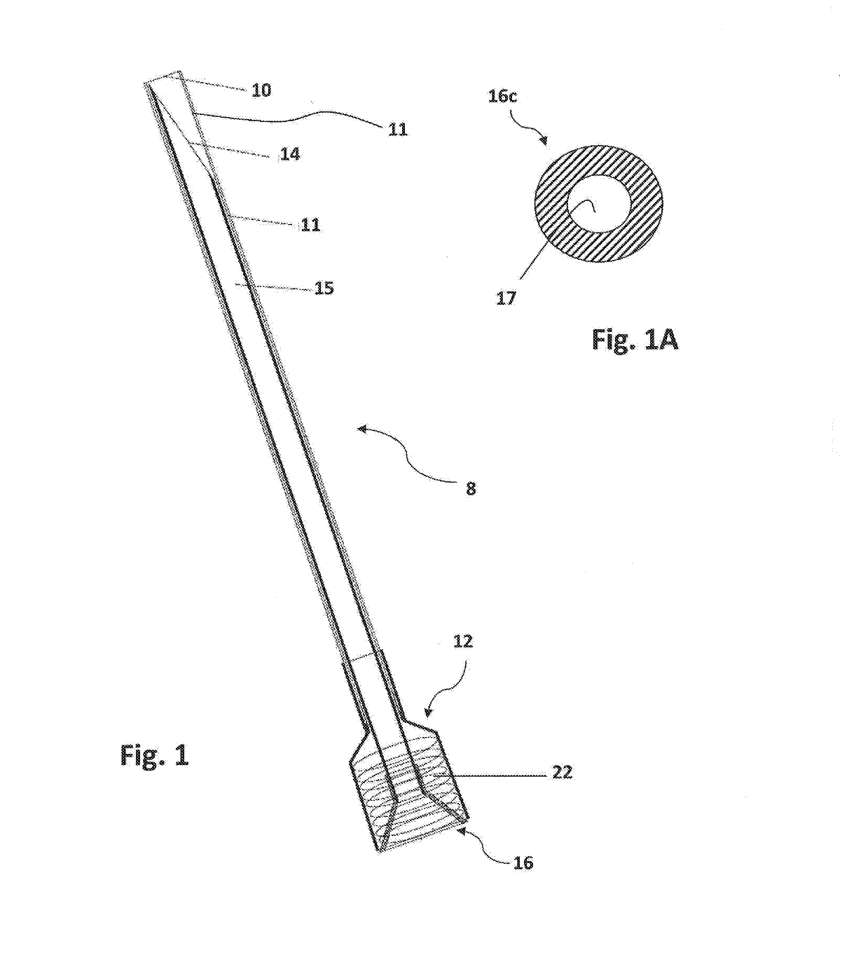

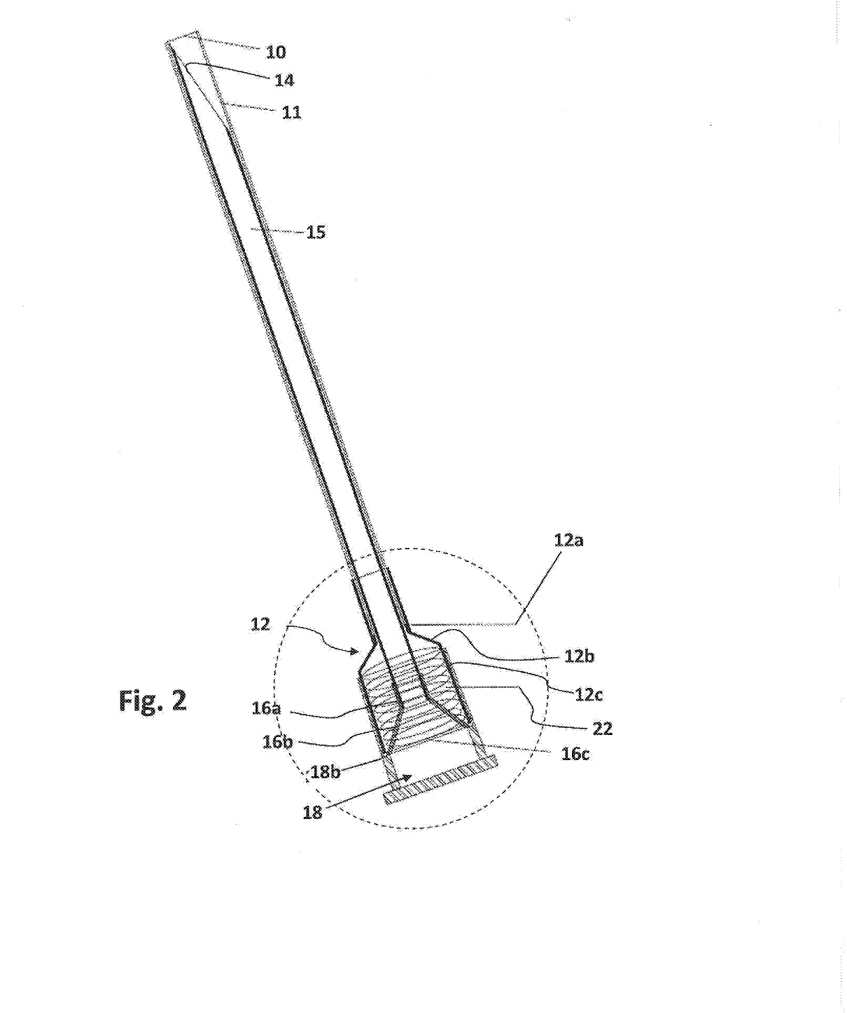

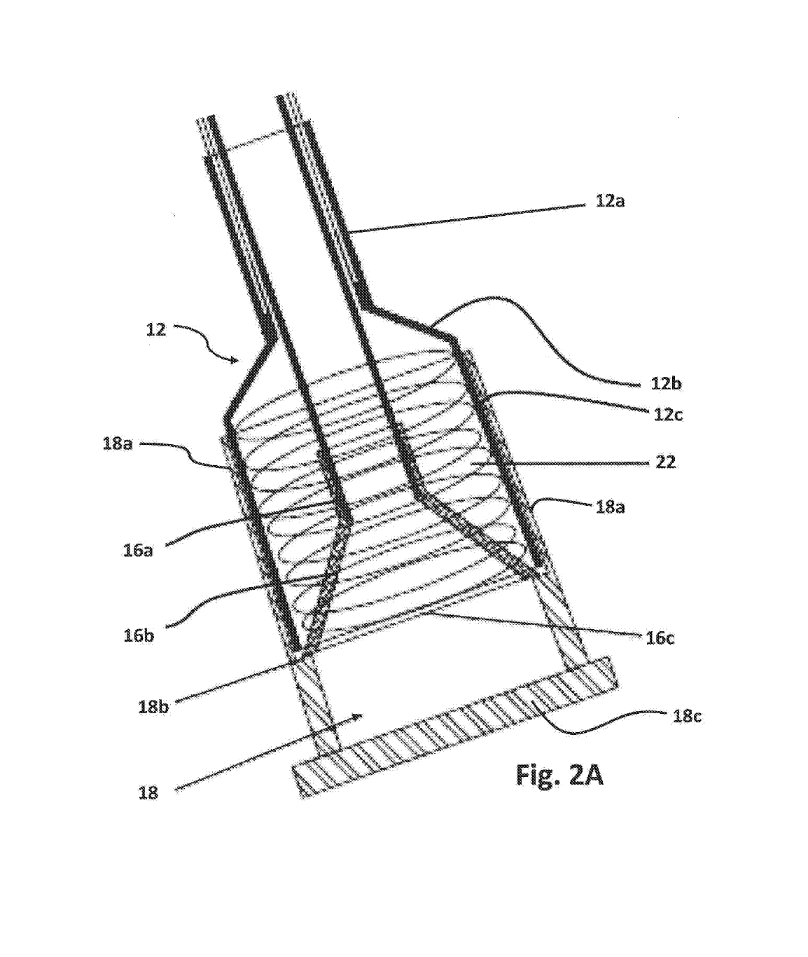

Referring to FIGS. 1-3, the catheter introducer assembly 8 comprises an outer tube 11, an outer tube housing 12, a needle 15, a needle extension member 16, a spring 22, and a retainer portion 18 (FIGS. 2, 2A & 3). The outer tube 11 comprises a blunt distal end 10. At a proximal end, the outer tube 11 is coupled to the outer tube housing 12. Referring to FIG. 2, in the preferred embodiment, the outer tube housing 12 comprises a tubular funnel-shaped configuration such that the outer tube housing 12 comprises a narrow distal portion 12a, a tapering mid-portion 12b, and a wider proximal portion 12c. In the preferred embodiment, the narrow distal portion 12a comprises an inside diameter slightly larger than an outside diameter of the outer tube 11 such that the proximal end of the outer tube 11 nests within the narrow distal portion 12a of the outer tube housing 12. In the preferred embodiment, the outer tube 11 is glued to the outer tube housing 12. However, the outer tube 11 may be coupled to the outer tube housing 12 in other ways. For example, the outer tube 11 may be frictionally engaged with the outer tube housing 12. In other embodiments, the outer tube 11 and outer tube housing 12 are formed from a single unbroken piece of material such that the outer tube 11 and outer tube housing 12 are a single piece rather than two pieces joined together. In other embodiments, the outer tube housing 12 narrow distal portion 12a nests within the outer tube 11 proximal end.

Nesting within the outer tube 11 and outer tube housing 12 are the needle 15, needle extension member 16, and the spring 22. The needle 15, is generally tubular, and comprises a sharp distal end (needle tip 14) and a blunt proximal end. A portion of the needle 15 proximate to the blunt proximal end of the needle 15 is coupled to the needle extension member 16. The needle extension member 16 is tubular and comprises a needle extension member tapering portion 16b and needle extension member retaining portion 16a. The needle extension member tapering portion 16b comprises a widened proximal end and a narrow distal end such that the needle extension member tapering portion 16b narrows in diameter between the proximal end of the widened needle extension member tapering portion 16b and the needle extension member retaining portion 16a. The needle extension member retaining portion 16a comprises an inside diameter slightly larger than en outside diameter of the needle 15 such that the proximal end of the needle 15 nests within the needle extension, member retaining portion 16a.

In the preferred embodiment, the needle extension member 16 is glued to the proximal end of the needle 15. However, the need e extension member 16 may be coupled to the needle 15 in other ways. For example, the needle extension member 16 may be frictionally engaged with the needle 15. In other embodiments, the needle extension member 16 and needle 15 are formed from a single unbroken piece of material such that the needle extension member 16 and needle 15 are a single piece rather than two pieces joined together. In other embodiments, the needle extension member retaining portion 16a nests within the needle 15.

As will be discussed in more detail below, the proximal end of the needle extension member 16 comprises a push plate 16c. Push plate 16c spans between perimeter walls of the needle extension member 16 and serves as a surface against which a male luer 30 can push so as to cause the needle tip 14 to extend beyond the distal end of outer tube 11. In some embodiments push plate comprises a through opening 17. This opening 17 permits fluids to pass through the push plate, when, for example, a syringe is connected to the assembly 8. The proximal end of the needle extension ember 16 comprises an outside diameter that is slightly smaller than an inside diameter of the outer tube cap wider proximal portion 12c. Thus, the needle 15, together with the attached needle extension member 16 can freely move, coaxially, within the outer tube housing wider proximal portion 12c. The spring 22 of the preferred embodiment is a coil spring 22. The spring 22 is positioned around the perimeter of the needle extension member 16 and a portion of the proximal end of the needle 15 and extends between the outer tube housing wider proximal portion 12c and the tapering mid-portion 12b of the outer tube housing 12. When in position, the spring 22, needle 15, needle extension member 16, outer tube 11 and outer tube housing 12 are coaxial.

Referring to FIGS. 2, 2A, & 3, the retainer portion 18 is coupled to the outer tube housing 12. The retainer portion 18 is generally tubular in configuration and comprises retainer portion wall 18a and retainer rim 18c. Retainer wall 18a is cylindrical and forms a retainer portion distal inside diameter that is slightly larger than the outside diameter of the wider proximal portion 12c of outer tube housing 12 such that the outer tube housing 12 nests within a distal section of the retainer portion 18.

In the preferred embodiment, the retainer portion 18 is glued to the wider proximal portion 12c of outer tube housing 12b. However, the retainer portion 18 may be coupled to the outer tube housing 12 in other ways. For example, the retainer portion 18 may be frictionally engaged with the wider proximal portion 12c of outer tube housing 12. In other embodiments, the retainer portion 18 and outer tube housing 12 are formed from a single unbroken piece of material such that the retainer portion 18 and outer tube housing 12 are a single piece rather than two pieces joined together. In other embodiments, the retainer portion 18 nests within the outer tube housing 12.

The retainer portion wall 18a comprises a retainer portion proximal inside diameter that is smaller than the inside diameter of the outer tube housing 12. This retainer portion proximal inside diameter is created by thickened walls 18a in an area distal to the outer tube housing 12. The thickened portions of walls 18a form a ridge 18b against which the push plate 16c rests when the retracted rest position depicted, for example, in FIGS. 2 and 2A. The ridge 18b prevents the needle extension member from exiting the proximal end of the assembly 8.

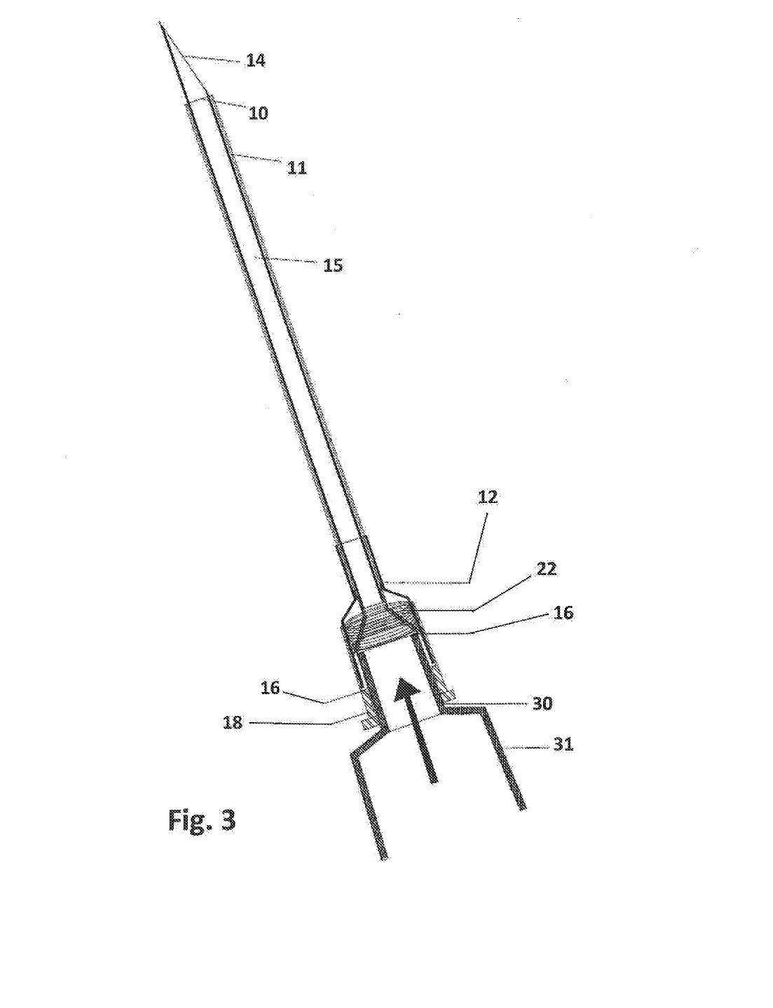

Referring to FIG. 3, the retainer portion 18 forms a female luer hub adapted to receive a male luer slip 30 of a syringe barrel 31. In use, the male luer slip 30 is pressed towards the push plate 16c between the walls 18a of the retainer portion 18. This action causes the needle tip 14 to extend beyond the distal end of outer tube 11.

Thus the catheter introducer assembly 8 of the present invention offers automatic protrusion of the needle tip 14 out of the outer tube 11 once the syringe barrel 31 is connected to the assembly 8. The needle tip 14 automatically retracts back into the outer tube 11 once the syringe barrel 31 is disconnected.

The preferred materials of construction of the outer tube 11, outer tube housing 12, needle extension member 16, and retainer portion 18 are PVC and polycarbonate. The needle 15 may be a conventional and commercially available needle 15, or may be a specially formed unitary needle/needle extension member 15, 16. The needle 15 is preferably stainless steel while the needle extension member is polypropylene, PVC or polycarbonate.

The spring 22 is a conventional and commercially available spring 22 having dimensions which permit it to be inserted within the outer tube housing 12 as disclosed herein. However, while certain materials are discussed herein with respect to the components of the assembly 8, the assembly 8 is not limited to such materials. Portions of the assembly 8, such as the outer tube 11, may comprise polyurethane, silicone or like material (e.g., a soft plastic or elastomer).

In the preferred embodiment of the safety guidewire introducer assembly 8, a luer connection between a male luer slip 30 that can be coupled to a female luer 18 formed by the retainer portion 18 for percutaneous entry into a body artery is provided. Although the safety guidewire introducer assembly 8 of the preferred embodiment comprises a modified luer connector 18, 30, the connector 18, 30 need not be a luer type connector 18, 30. Rather, other connectors 18, 30 may be modified in accordance with this specification without departing from the scope and spirit of this disclosure. For example, in other embodiments, the connector 18, 30 may comprise barbs or ridges on an outer surface.

The foregoing disclosure and showings made in the drawings are merely illustrative of the principles of this invention and are not to be interpreted in a limiting sense. While the invention is shown in only a few forms, it is not just limited to the forms shown, but is susceptible to various changes and modifications without departing from the spirit thereof. The foregoing description of a preferred embodiment of the invention has been presented for the purpose of illustration and description. It is not intended to be exhaustive or to limit the invention to the precise form disclosed. Obvious modifications or variations are possible in light of the, above teachings. The invention may be adapted for use in a number of environments.

The embodiments were chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention in accordance with the breadth of this disclosure and the appended claims, to which they are fairly, legally, and equitably entitled to be interpreted.

1. A safety guidewire introducer assembly comprising:
a needle, an extension member, an outer tube, a housing portion, a spring and a retainer portion;
the needle and outer tube each comprising respective first and second ends;
the needle first end comprising a needle tip, the needle second end being coupled to the extension member;
the extension member comprising a receiving member, an expanded portion, and a plate member;
the receiving member being coupled to the needle second end;
the expanded portion being coupled to the plate member;
the outer tube comprising outer tube first and second ends;
the outer tube second end being coupled to the housing portion, the housing portion comprising distal, intermediate, and proximal portions;
the distal portion being coupled to the outer tube second end, the intermediate portion spanning between the distal and proximal portions;
the proximal portion comprising an inside diameter, said inside diameter being greater than an expanded portion outside diameter;
the spring being positioned within the proximal portion of the housing and comprising a coiled configuration and having a spring outside diameter which is less than the proximal portion inside diameter;
the spring outside diameter being less than or equal to the expanded portion outside diameter such that the needle, extension member, and spring nest coaxially within the housing portion; and
the housing portion being coupled to said connection port, said retainer portion structured and arranged to retain the extension member within the housing portion.

2. The safety guidewire introducer assembly of claim 1, the expanded portion comprising a conical configuration.

3. The safety guidewire introducer assembly of claim 1, the intermediate portion comprising a conical configuration.

4. The safety guidewire introducer assembly of claim 1, wherein the receiver portion is glued to the needle, the housing is glued to the outer tube, and the retainer portion is glued to the housing.

5. The safety guidewire introducer assembly of claim 1, the retainer portion being adapted to receive a male luer slip.

6. The safety guidewire introducer assembly of claim 1, wherein the needle tip is retained within the outer tube when the needle is in a first position and the needle tip extends beyond the outer tube first end when the needle is in a second position.

7. The safety guidewire introducer assembly of claim 6, wherein the needle tip is spring biased in the first position.

8. The safety guidewire introducer assembly of claim 7, wherein the needle tip is moved from the first position to the second position upon coupling of a male luer slip to the retainer portion.

9. The safety guidewire introducer assembly of claim 8, wherein the needle tip is moved from the second position to the first position upon decoupling of the male luer slip from the retainer portion.

10. The safety guidewire introducer assembly of claim 1, herein the plate member comprises an opening.

11. The safety guidewire introducer assembly of claim 1, the retainer portion comprising an inner ridge.

12. The safety guidewire introducer assembly of claim 1, the retainer portion comprising an inside diameter that is less than the outside diameter of the extension member.

13. A safety guidewire introducer assembly comprising:
a needle, an extension member, an outer tube comprising, a housing portion, a spring and a retainer portion;
the needle and outer tube each comprising respective first and second ends;
the needle first end comprising a needle tip, the needle second end being coupled to the extension member;
the extension member comprising a receiving member, an expanded portion, and a plate member:
the receiving member being coupled to the needle second end;
the expanded portion being coupled to the plate member, the plate member comprising a central opening;
the outer tube comprising outer tube first and second ends;
the outer tube second end comprising the housing portion, the housing portion comprising distal, intermediate, and proximal portions;
the distal portion being coupled to the outer tube second end, the intermediate portion spanning between the distal and proximal portions;
the proximal portion comprising an side diameter, said inside diameter being greater than an expanded portion outside diameter;
the spring being positioned within the proximal portion of the housing and comprising a coiled configuration and having a spring outside diameter which is less than the proximal portion inside diameter;
the spring outside diameter being less than or equal to the expanded portion outside diameter such that the needle, extension member, and spring nest coaxially within the housing portion;
the expanded portion comprising a conical configuration;
the intermediate portion comprising a conical configuration;
the housing portion being coupled to said retainer portion, said retainer portion comprising an inner ridge structured and arranged to retain the extension member within the housing portion and being adapted to receive a male leer slip;
wherein the needle tip is retained within the outer tube when the needle is in a first position and the needle tip extends beyond the outer tube first end when the needle is in a second position, the needle tip being spring biased in the first position.

14. The safety guidewire introducer assembly of claim 13, wherein the needle tip is moved from the first position to the second position upon insertion of a male luer slip within the retainer portion and wherein the needle tip is moved from the second position to the first position upon decoupling of the male luer slip from the retainer portion.

15. The safety guidewire introducer assembly of claim 14, wherein the male luer is a syringe male luer and upon coupling the receiver portion is pushed towards the intermediate portion by the syringe male luer causing the spring to compress and upon decoupling, the receiver portion is pushed away from the intermediate portion by the spring.
